(19) United States
(12) Patent Application Publication
(10) Pub. No.: US 2010/0332149 A1
(43) Pub. Date: Dec. 30, 2010

(54) METHOD AND SYSTEM FOR REMOTE MONITORING OF FLUID QUALITY AND TREATMENT

(75) Inventor: Charles SCHOLPP, Westminster, CO (US)

Correspondence Address: Vedder Price P.C., 875 15th Street NW, Suite 725, Washington, DC, 20005, US

(73) Assignee: Hach Company, Loveland, CO (US)

(21) Appl. No.: 12/874,293

(22) Filed: Sep. 2, 2010

Related U.S. Application Data
(63) Continuation of application No. 12/785,549, filed on May 24, 2010, now abandoned.
Application No. 12/785,549 is a continuation-in-part of application No. 12/710,451, filed on Feb. 23, 2010.
Application No. 12/710,451 is a division of application No. 12/272,018, filed on Nov. 17, 2008, now Pat. No. 7,698,073.
Application No. 12/272,018 is a continuation of application No. 10/392,112, filed on Mar. 19, 2003, now Pat. No. 7,454,295.
Application No. 10/392,112 is a continuation-in-part of application No. 10/055,225, filed on Oct. 26, 2001, now Pat. No. 6,560,543.
Application No. 10/055,225 is a continuation-in-part of application No. 09/213,781, filed on Dec. 17, 1998, now Pat. No. 6,332,110.
Application No. 09/213,781 is a continuation-in-part of application No. 12/565,091, filed on Sep. 23, 2009.
Application No. 12/565,091 is a continuation-in-part of application No. 11/331,721, filed on Jan. 13, 2006.
Application No. 11/331,721 is a continuation-in-part of application No. 10/695,627, filed on Oct. 27, 2003, now Pat. No. 6,954,701.
Application No. 10/695,627 is a continuation-in-part of application No. 10/392,112, filed on Mar. 19, 2003, now Pat. No. 7,454,295.
Application No. 10/392,112 is a continuation-in-part of application No. 11/331,721, filed on Jan. 13, 2006.

(51) Int. Cl. G06F 19/00; G01N 33/18; G06F 15/00

(52) U.S. Cl. 702/25; 702/188

(57) ABSTRACT

The present invention provides a remote monitoring system for monitoring the operation of a fluid treatment system and/or the qualities, characteristics, properties, etc., of the fluid being processed or treated by the fluid treatment system. The system for measuring fluid quality and/or equipment operation in a fluid treatment system includes a remote computer that may be associated with a database that accesses data transmitted from the fluid treatment system with the data collected, acquired, etc., from one or more sensors placed in the fluid treatment system. The data may be analyzed or manipulated by a local computer and/or the remote computer, which may be used to generate an analysis result or analysis report. Such results or reports, along with any other information or data including historical or expected information, may be sent or communicated to a remote viewing device for viewing by a user. Methods are further provided for the operation of the remote monitoring system of the present invention.

### CROSS-REFERENCE TO RELATED APPLICATIONS

This application is a continuation-in-part of U.S. patent application Ser. No. 12/710,451, filed Feb. 23, 2010, which is a divisional of U.S. patent application Ser. No. 12/272,018 filed Nov. 17, 2008, now U.S. Pat. No. 7,698,073, which is a continuation of U.S. patent application Ser. No. 10/392,112 filed Mar. 19, 2003, now U.S. Pat. No. 7,454,295, which is a continuation-in-part of U.S. patent application Ser. No. 10/055,225 filed Oct. 26, 2001, now U.S. Pat. No. 6,560,543, which is a continuation-in-part of U.S. patent application Ser. No. 09/213,781 filed Dec. 17, 1998, now U.S. Pat. No. 6,332,110. This application is also a continuation-in-part of U.S. patent application Ser. No. 12/565,091 filed Sep. 23, 2009, which is a continuation-in-part of: U.S. patent application Ser. No. 11/331,721 filed Jan. 13, 2006; U.S. patent application Ser. No. 12/272,018; and U.S. patent application Ser. No. 10/695,627 filed Oct. 27, 2003, now U.S. Pat. No. 6,954,701. U.S. patent application Ser. No. 10/695,627 is a continuation-in-part of U.S. patent application Ser. No. 10/392,112, and U.S. patent application Ser. No. 11/331,721 is a continuation-in-part of U.S. patent application Ser. No. 12/272,018. This application is also a continuation-in-part of U.S. patent application Ser. No. 11/331,721. The entire contents and disclosures of each of the above applications/patents are hereby incorporated by reference.

### FIELD OF THE INVENTION

The present invention relates to the field of fluid treatment and safety, and in particular, to a method and system allowing remote monitoring and/or storage of fluid treatment and safety data.

### BACKGROUND

It is well recognized that many aspects of manufacturing, as well as life itself, is dependent upon water. Water may be characterized by the amount of cations and anions, metals, turbidity, dissolved solids, and so forth, all of which combine to form unique water chemistries. Technology provides the ability to adjust, reduce, or remove such qualities to effectively prepare water for use in a particular application. Proper water treatment systems provide an economical way of conditioning water to a predetermined quality level as required for the particular application. Protection of water supplies from system or equipment failure as well as inadvertent or deliberate contamination are important concerns. While devices and methods exist to analyze water for contaminants, widespread deployment of such devices is expensive and difficult.

Many processes and applications require the use of water having sufficiently low or absent levels of contaminants or harmful substances, and thus rely on the use of water treatment systems to ensure adequate levels of water purity, quality, and/or safety. These water treatment systems may generally use techniques, such as advanced separation, filtration, reverse osmosis, and/or ion exchange processes, as well as the introduction of materials or disinfectants to achieve the desired water quality. However, equipment failure or tampering of these systems may result in poor or unsafe water quality for a given application. Therefore, it is critical that any water treatment system used to purify or treat water for any such applications is adequately monitored to ensure that the desired levels of water purity, quality, and/or safety are met. One application in which water quality is important is in providing potable drinking water to the public. Most water treatment systems for the production and distribution of drinking water to the public rely, for example, on the introduction and maintenance of materials, such as disinfectants, into the water system to protect against biological or chemical contamination. Chlorine, in the form of gas or hypochlorite or hypochlorous acid, is one of the most common materials used for this purpose. Substitutes such as chloramines, ozone, hydrogen peroxide, peracetic acid, chlorine dioxide, and various mixed oxides are also used. Many of these materials have a more or less common mode of action. They rely on some sort of oxidation to effect the deactivation of biological organisms and the destruction of other organic compounds present in the water to be treated. The reaction rates of the various materials, such as disinfectant compounds, are reasonably well known and well characterized. However, excessive amounts of these materials may cause problems on their own. Thus, it is important that adequate monitoring is performed to ensure that sufficient but not excessive amounts of these materials or disinfectants are maintained in a water treatment system.

Municipal drinking water may be obtained from a variety of sources, which can be made potable by use of proper water treatment equipment. For example, a reverse osmosis system may be used to lower the total dissolved solids from sea water with minimal pretreatment to produce potable drinking water. Despite the sophistication of pretreatment of seawater, improper monitoring or operation can allow the seawater to quickly foul membranes. If fouling occurs, but is found quickly, the membranes may be cleaned, and water contamination and associated water treatment repairs may be averted. However, if the fouling is not detected quickly through proper monitoring, the membranes can be irreparably damaged, and expensive partial or total membrane replacement would be required. The cost of unplanned membrane replacement, not including the lost revenues typically associated with down time, can make such a system cost prohibitive.

Another application in which water quality is important is with Waste Water Treatment Plants (WWTP). The treatment and subsequent recycling of wastewater is a cornerstone of the quality of life in the industrialized world. Cities, industries, and agricultural operations produce large quantities of wastewater, all of which must be treated to some degree to remove contaminants or pollutants before the water is suitable for recycling or discharge into the environment, such as streams, rivers or oceans. In metropolitan areas, central waste water treatment plants must treat water from a variety of sources including city, industrial, and agricultural waste water. In many cases, generators of industrial waste water are required to install and operate waste water treatment plants at their own sites before discharge into central water collection systems. At the central water collection system, industrial wastes may generally be mixed with domestic or city waste water and other untreated waste sources. These mixed wastes are then transported to the central waste water plant or sewage treatment facility for final treatment before discharge.

Increasingly, the need for pure water is causing more and more municipalities to install waste water recovery processes to recycle municipal WWTP effluents back into water of suitable quality to be used for potable drinking water or irrigation. For example, such recovery processes may recover secondary treated municipal effluents using reverse osmosis, which may then be injected back into an aquifer. More and more of these installations are planned throughout the United States and the rest of the world.

One difficult aspect of treating municipal waste water effluent is that neither the flow rates nor the mix of contaminants are constant. This is particularly true for a municipal WWTP with collection systems that include a variety of industrial discharge sources in addition to the usual sanitary discharges from homes, businesses, schools, and so on. While the sanitary discharges are well characterized in terms of composition and treatability, the addition of industrial wastes means that the WWTP must plan for a wide variety of contaminants. In general, most WWTP systems cannot deal effectively with every situation. Even with excellent design and engineering, the large fluctuation in the type and quantity of contaminants reaching the WWTP often results in varying levels of effective treatment in the discharge from the WWTP. For a tertiary water recovery plant treating the effluent from the WWTP this can be particularly difficult since many contaminants are not readily removed even by processes such as reverse osmosis. In addition, certain contaminants can also foul reverse osmosis, ultrafiltration, and microfiltration membranes, causing loss of performance or membrane damage. Therefore, it is important that WWTPs are monitored to ensure that contaminants are properly removed before discharge or reuse back into the environment and to avoid damage to expensive equipment.

Although systems exist for the local monitoring of discrete, independent treatment site locations for individual analysis, these systems do not contemplate remote monitoring of one or a number of water treatments sites throughout a collection system that simultaneously feed effluents into a central water collection system of a WWTP. There remains a need for a system designed for remote monitoring of a WWTP which may collect and interpret data from one or a multiple number of remote industrial or water treatment sites viewed and analyzed as an aggregate water treatment system.

Water is also required for steam generation in nuclear reactors. The boilers of these nuclear reactors operate at extremely high temperatures which require a very high quality of water. It is critical that the process system is monitored properly to avoid expensive boiler cleanings and the associated down time. Such systems may also include the need to monitor hazardous boiler chemicals, such as hydrazine, requiring highly qualified personnel. These examples highlight the importance of monitoring the operation of water treatment systems to not only ensure sufficient water quality, but also to avert costly equipment repair or replacement.

Water quality is also important for many manufacturing processes. For example, the manufacturing of semiconductors requires an ultra-pure water quality. Again, it is critical that the water treatment system is monitored properly to avoid latent defects in the manufacturing of products, such as semiconductors.

As yet another example, monitoring water quality is also important to avoid or lessen the consequences of equipment failure or deliberate tampering, such as by terrorist act, in contaminating the water supply. Adequate monitoring may help to catch any such contamination of the water supply to avoid harm and ensure that appropriate action is taken.

One of the problems with maintaining advanced processing equipment is the need for highly qualified individuals to monitor its operation. Employment of a full time staff is costly and can be problematic since such monitoring is repetitive, and highly qualified individuals can easily become bored or distracted. For this reason, advanced separation processes may include a large assortment of strategically placed sensors that are typically incorporated into a computer system capable of comparing the sensor values against a pre-set quality level. However, if the operator is not notified or does not recognize an abnormal condition, the elaborate array of monitoring equipment is effectively useless.

### SUMMARY

According to a first broad aspect of the present invention, a remote monitoring system is provided comprising one or more sensors located within a water treatment system being monitored; a remote computer disposed at a first distant location from the water treatment system; and an analyzer for manipulating data obtained from the one or more sensors of the water treatment system, wherein the data is transmitted from the water treatment system to the remote computer using a mode of transmission, and wherein the remote computer generates an output from the manipulated data.

According to a second broad aspect of the present invention, a method is provided comprising the following steps: (a) transmitting data collected from one or more sensors in the water treatment system to a remote computer disposed at a first distant location from the water treatment system; and (b) generating an output based on the data, wherein the data is transmitted from the water treatment system to the remote computer using a mode of transmission. According to some embodiments, the remote computer is only connected or linked to the water treatment system or to sensors, electronic control system, and/or local computer located within the water treatment system via the mode of transmission. Some embodiments of the second broad aspect may further comprise the step of (c) manipulating the data using an analyzer. According to some embodiments, the manipulating step (c) may be performed after step (a). According to these embodiments, the analyzer may be located at a second distant location from the water treatment system, and the first and second distant locations may be co-located, such as with the analyzer associated with or located on the remote computer. Alternatively, the manipulating step (c) may be performed prior to step (a). According to these embodiments, the data may be transmitted from a local computer located at or near the water treatment system to the remote computer during step (a). According to some embodiments, the local computer may be a logger device. Some embodiments of the second broad aspect may further comprise the step of (d) communicating the output to a remote viewing device using a mode of communication, and step (d) may be performed after step (b). In addition, method embodiments of the second broad aspect may further comprise the step of (e) storing the data on a remote database associated with the remote computer, and step (e) may be performed after step (b).

According to a third broad aspect of the present invention, a method is provided comprising the following steps: (a) collecting data from one or more sensors located in the water treatment system; and (b) transmitting the data to a remote computer disposed at a first distant location from the water treatment system using a mode of transmission. Some embodiments of the third broad aspect may further comprise the step of (c) generating an output based on the data, which may be performed after step (b). Some embodiments of the third broad aspect may further comprise the step of (d) communicating the output to a remote viewing device using a mode of communication, and step (d) may be performed after step (b). Some embodiments of the third broad aspect may further comprise the step of (e) manipulating the data using an analyzer. According to some embodiments, step (e) may be performed prior to step (b), and the analyzer may be associated with a local computer. According to some alternative embodiments, step (e) may be performed after step (b), and the analyzer may be associated with the remote computer.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a diagram of an embodiment of the remote monitoring system of the present invention; and

FIG. 2 is a diagram of an embodiment of the remote monitoring system of the present invention with a local computer.

### DETAILED DESCRIPTION

### Definitions

Where the definition of terms departs from the commonly used meaning of the term, applicant intends to utilize the definitions provided below, unless specifically indicated.

For purposes of the present invention, the term “water treatment system” refers to any system designed or used to process, treat, or generate water or a water-based product for a particular application. A “water treatment system” may be used to generate water having a predetermined, desired, or preferred set of characteristics, qualities, or properties, such as purity, etc. For example, a “water treatment system” may include a water treatment facility for generating and distributing potable drinking water for the public, a system designed to generate water for a manufacturing process, etc. In the case of a water treatment facility for generating potable drinking water, the water treatment system may further include a distribution system for distributing the potable drinking water to the public. A “water treatment system” may also be any system used to process or treat a water-based substance into a product that may be discharged into the environment, such as, for example, a central wastewater treatment plant (WWTP), etc. In the case of a WWTP, the water treatment system may further include a collection system for collecting waste water and funneling it into the central WWTP. Water treatment systems may include public or municipal systems or private systems dedicated to an industry, factory, or particular real estate development. For example, a water treatment system may include any system, plant, or facility that uses equipment based on advanced separation, filtration, dialysis, ion exchange processes, or any other basis, technology, or mechanism for processing, treating, detecting, purifying, isolating, separating, etc., water according to relevant parameters.

For purposes of the present invention, the term “water treatment core facility” refers to a central facility that processes, treats, generates, etc., water in contrast to a broader collection or distribution system, such as a central wastewater treatment plant (WWTP), for the processing or treatment of waste water, or a water treatment facility, such as a facility for the generation of potable drinking water.

For purposes of the present invention, the term “water” refers to water or any fluid that may be processed, treated, generated, produced, discharged, etc., by a water treatment system. For example, the term “water” may refer to water being treated or processed by a water treatment facility for the distribution of potable drinking water to the public, or the term “water” may refer to sewage or waste water processed or treated by a central wastewater treatment plant (WWTP). Thus, “water” may include any number of solutes, sediments, suspensions, organic matter, etc., as the case may be.

For purposes of the present invention, the terms “treat,” “treated,” “treating,” “treatment,” and the like shall refer to any process, treatment, generation, production, discharge, or other operation that may be performed by a water treatment system on, or in relation to, the water in the water treatment system.

For purposes of the present invention, the term “remote monitoring system” refers to a system for remotely monitoring the operation and equipment of a non co-located water treatment system or the water quality in, toward, or from a non-collocated water treatment system using sensors to collect data that is transmitted to a remote computer for analysis, manipulation, and communication to a remote viewing device for a user.

For purposes of the present invention, the term “user” refers to a person, entity, or agency that views data, information, analysis results, or analysis reports communicated from the remote computer to the remote viewing device of the present remote monitoring system.

For purposes of the present invention, the term “sensor” refers to a device, probe, or apparatus for the detection or measurement of parameters or values relevant to water quality or the operation of a water treatment system. The term “sensor” may refer to a device, probe, or apparatus connected to a local computer, such as a logger device.

For purposes of the present invention, the term “electronic control system” refers to a portion of a water treatment system that may control the operation of equipment and operation of a water treatment system. According to some embodiments, a remote computer of the present invention may access or collect data from one or more sensors via an electronic control system. An electronic control system may include an in-house Supervisory Control and Data Acquisition System (SCADA) or a Progammable Logic Controller (PLC).

For purposes of the present invention, the term “data” refers to any information, reading, measurement, value, etc., ultimately obtained from one or more sensors or derived from such data. The term “data” includes any data or information including raw data obtained directly from one or more sensors without manipulation, historical data earlier obtained from one or more sensors or entered or derived from data obtained at an earlier point or period in time, and analyzed or manipulated data, such as data or information manipulated, analyzed, etc., by an analyzer. The term “data” may include, for example, an analysis result or observational data.

For purposes of the present invention, the term “remote computer” refers to an electronic device of the present remote monitoring system that is capable of storing, processing, and/or manipulating data, raw data or historical data, such as a computer, server, etc., that is physically separated, i.e., at a remote or distant location, from the location of the water treatment system monitored by such system. For example, a “remote computer” may include a web or Internet server. The “remote computer” may further include a database and/or an analyzer.

For purposes of the present invention, the term “remote database” refers to a device or apparatus of the present remote monitoring system used to store data, raw data, historical data, manipulated data and/or information, such as in a logical or ordered arrangement or configuration. The remote database may be part of the remote computer or separate, albeit connected to or in communication with, the remote computer. As such, the “remote database” is physically separated, i.e., at a remote or distant location, from the location of the water treatment system.

For purposes of the present invention, the term “distant” in reference to a remote computer and/or remote database refers to the remote computer and/or remote database being physically separated from a water treatment system. The term “distant” may refer to the remote computer and/or remote database being located away from the premises of a water treatment system and/or a water treatment core facility. The term “distant” may refer to a remote computer and/or remote database that is only connected or linked to a water treatment system (or only connected or linked to the one or more sensors, electronic control system, and/or local computer located within the water treatment system) via a mode of transmission.

For purposes of the present invention, the term “analyzer” refers to a portion of the local computer or the remote computer of the present remote monitoring system which may be stored on the local computer and/or the remote computer, such as a software program(s) or other routine(s), firmware, and/or hardware, which may analyze, manipulate, etc., data, raw data, observational data, historical data, or any other information obtained from one or more sensors. When the local computer is a logger device, the “analyzer” may be located on the logger device.

For purposes of the present invention, the term “local computer” refers to any type of computer, processor, or device physically located at or near a water treatment system (i.e., not remotely located) and connected to the one or more sensors either directly or indirectly. The local computer may assemble, collect, aggregate, manipulate, or analyze data from one or more sensors of the present remote monitoring system prior to the data being transmitted to the remote computer of the present remote monitoring system. The “local computer” may be any computer, etc. able to (1) at least temporarily store, assemble, collect, aggregate, etc., data from one or more sensors and (2) transmit data or information to a remote computer (or a remote database associated with the remote computer) via a mode of transmission. Thus, a “local computer” may contain or include (1) a memory device(s) to store, assemble, collect, aggregate, etc., the data at least temporarily, (2) one or more ports or inputs for receiving data or information either directly or indirectly from one or more sensors, and (3) a transmission interface(s) to transmit data or information to a remote computer. A “local computer” may further have the ability to process, manipulate, analyze, etc., the data obtained from the one or more sensors, such as by an analyzer or software located on local computer, prior to transmission of data or information to the remote computer and/or remote database. The “local computer” may be a logger device as described herein.

For purposes of the present invention, the term “observational data” refers to data or information that has been analyzed, manipulated, etc., by the local computer, such as by an analyzer on the local computer, from raw data or information obtained from one or more sensors prior to being transmitted to a remote computer and/or remote database.

For purposes of the present invention, the term “remote viewing device” refers to any device or apparatus known in the art that may be used to view an output of the present remote monitoring system from the remote computer, such as, for example, personal computers or terminals, servers, etc., as well as a variety of handheld personal communications equipment, such as cell phones, pagers, PDA's, Blackberrys®, Palm® devices, iPhones®, etc.

For purposes of the present invention, the term “output” refers to any product, publication, submission, uploaded content, etc., including any information, data, analysis result, analysis report, etc., that may be communicated from the remote computer of the present remote monitoring system to a remote viewing device in a format suitable for display by the remote viewing device to a user.

For purposes of the present invention, the term “analysis result” refers to any information, value, relationship, product, etc., created by aggregation, calculation, algorithm, analysis, manipulation, etc., of data or information obtained or collected from one or more sensors as performed by an analyzer on the local computer and/or the remote computer of the present remote monitoring system. For example, an “analysis result” may include observational data analyzed, manipulated, etc., by a local computer.

For purposes of the present invention, the term “analysis report” refers to any organized presentation of data, raw data or historical data, manipulated data, observational data, information, analysis result, etc., based on data obtained or collected from one or more sensors that is generated or manipulated by an analyzer on the remote computer of the present remote monitoring system. An analysis report may be prepared for any intended recipient, such as an elected official, manager or operator of a water treatment system, customer, member of the public, etc. According to some embodiments, an “analysis report” may be a submission to a regulatory and/or law enforcement agency in any required format.

For purposes of the present invention, the term “transmission interface” refers to a portion of a local computer, electronic control system, and/or one or more sensors of a remote monitoring system that is able of transmitting data or information to a remote computer via any suitable mode of transmission.

For purposes of the present invention, the term “mode of transmission” refers to any suitable technology or device known and available in the art for transmitting data and information to a remote computer of the present remote monitoring system. The data and information may be transmitted by the mode of transmission either directly from the one or more sensors, from an electronic control system, or from a local computer connected to the electronic control system and/or one or more sensors, which may each utilize a transmission interface. The mode of transmission may include any of the technologies used for the mode of communication. Examples of modes of transmission may be achieved or carried out through any suitable medium, such as any wired or wireless connections as well as any protocols, including, but not limited to: the Internet; TCP/IP; MODBUS RTU, MODBUS ASCII, and MODBUS TCP; XML; Ethernet; file transfer protocol (FTP); email, such as SMTP; cellular modem; Bluetooth®; ZigBee®; cellular phone networks, such as CDMA and TDMA; radio signals or remote terminal units (RTU) coupled to radio frequency transmitters; satellite transmission; SDI-12; existing telephone or communication networks or wiring, a standard Public Switched Telephone Network (PSTN); dial-up using landline or telephone; a wireless network, such as wi-fi; a wide area network (WAN); wireless local area network (WLAN); local area network (LAN); or metropolitan area network (MAN); a cable internet connection; short message system (SMS); dial-up modem; a point to point link; global system for mobile communications (GSM, 3GSM), general packet radio services (GPRS), evolution-data optimized (EV-DO), enhanced data rates for GSM evolution (EDGE), digital enhanced cordless telecommunications (DECT), integrated digital enhanced network (iDEN), universal mobile telecommunications systems (UMTS), advanced mobile phone systems (AMPS) or any other suitable means to transmit data to a remote computer known to those skilled in the art. The exact mode of transmission may vary depending on the circumstances. According to embodiments of the present invention, the mode of transmission may transmit data or information continuously, in real time, at periodic or selected intervals, on condition, or on demand by a user.

For purposes of the present invention, the term “mode of communication” refers to any suitable technology for sending, uploading, or communicating an output, including data, information, analysis results, analysis reports, alerts, alarms, etc., from a remote computer to a remote viewing device of the present remote monitoring system. The mode of communication may include any of the technologies used for the mode of transmission. For example, according to some embodiments, a suitable technology to serve as a “mode of communication” may be the Internet or world wide web. In such a case, the output may be uploaded onto an Internet server computer, which may be the remote computer of the present remote monitoring system or the Internet server computer may be separate from the remote computer. According to other embodiments, the “mode of communication” for sending an output to, or allowing access to an output by, a remote viewing device, includes, but is not limited to any wired or wireless connections as well as any protocols: the Internet; TCP/IP; MODBUS RTU, MODBUS ASCII, and MODBUS TCP; XML; Ethernet; file transfer protocol (FTP); Bluetooth®; ZigBee®; email, such as SMTP; cellular phone networks, such as CDMA and TDMA; radio signals or remote terminal units (RTU) coupled to radio frequency transmitters; cellular modem; SDI-12; satellite transmission; existing telephone or communication networks or wiring, a standard Public Switched Telephone Network (PSTN); a wireless network; a wide area network (WAN); wireless local area network (WLAN); local area network (LAN); or metropolitan area network (MAN); a cable internet connection; short message system (SMS); dial-up modem; a point to point link; global system for mobile communications (GSM, 3GSM), general packet radio services (GPRS), evolution-data optimized (EV-DO), enhanced data rates for GSM evolution (EDGE), digital enhanced cordless telecommunications (DECT), integrated digital enhanced network (iDEN), universal mobile telecommunications sytems (UMTS), advanced mobile phone systems (AMPS); or any other suitable means known to those skilled in the art to send, upload, or communicate an output to a remote viewing device.

### DESCRIPTION

Embodiments of the present invention provide a method and system for remotely monitoring, storing, analyzing, manipulating, uploading, reporting, etc., information and data relating to water quality and/or treatment derived from raw data obtained from a plurality of sensors of a water treatment system, which may be strategically placed to gather data or information necessary for analysis or manipulation. Such information and data may be remotely stored, manipulated, etc., on one or more remote computer(s), and/or stored on one or more remote database(s), which may be associated with the remote computer(s). A water treatment system according to embodiments of the present invention may include any system designed or used to generate water or a water-based product having a predetermined, desired, or preferred set of characteristics, qualities, properties, etc., for a particular application, such as, for example, a Municipal potable drinking water treatment facility, a system generating water for a manufacturing process, etc., as well as any distribution system. A water treatment system may also include any system designed or used to process or treat a water-based substance into a product discharged into the environment, such as, for example, a central wastewater treatment plant (WWTP), etc., as well as any collection system. Water treatment systems may include a public or municipal system as well as a system dedicated to a real estate development. For example, a water treatment system may include any system, plant, or facility that uses equipment based on advanced separation, filtration, dialysis, ion exchange processes, or any other basis, technology, or mechanism for processing, treating, detecting, purifying, isolating, separating, etc., water according to relevant parameters.

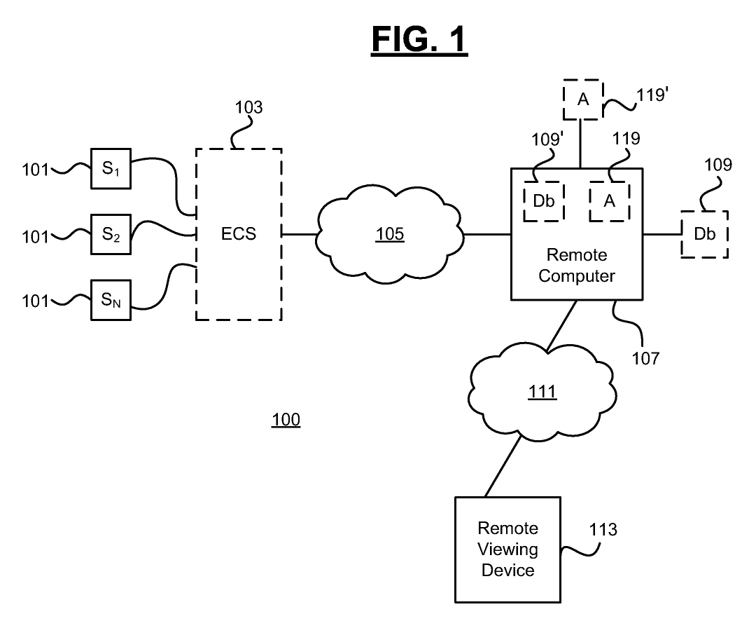

According to embodiments of the present invention as shown in FIG. 1, remote monitoring system 100 collects raw data from one or more sensors 101 located within a water treatment system and transmits such raw data to a remote computer(s) 107 via any known technology or mode of transmission 105. Although the embodiments shown in the figure depict data from sensors 101 being transmitted to remote computer 107 via an optional electronic control system (ECS) 103, it is to be appreciated that sensors 101 may transmit data directly to remote computer 107, which may occur in the absence of optional electronic control system (ECS) 103. According to some embodiments, remote computer 107 may be, for example, an Internet server computer. Remote computer 107 may store and/or manipulate raw data to produce an analysis result(s). Remote computer 107 may store data on a remote database 109′ may be located on remote computer 107 for storing the data. Alternatively, data may be stored by remote computer 107 on a remote database 109 associated with remote computer 107. The manipulation or analysis of data may be performed by an analyzer 119, 119′, which may be a software program, located on, associated with, or executed by remote computer 107. According to some embodiments, one or more sensors 101 may optionally transmit raw data to the remote computer 107 via an electronic control system 103, which may also control operation of the equipment of the water treatment system.

Once data is stored in either remote computer 107, remote database on remote computer 109′, and/or remote database 109, analyzer 119, 119′ on or executed by remote computer 107 may then analyze or manipulate data to generate manipulated data and/or an output including data and information, such as an analysis result(s) or analysis report(s), presenting or indicating the qualities, characteristics, properties, etc., of the water being treated and/or the operation of the water treatment system. The manipulation or analysis of data by analyzer 119, 119′ may be performed continuously, in real time, at periodic or selected intervals, on condition, or on demand for presentation to a user. Following analysis or manipulation by analyzer 119, 119′, the information, data, and/or analysis result(s) or report(s) may then be sent to a remote viewing device 113 using any known mode of communication 111. However, it is to be understood that according to some embodiments, raw data or direct readings may be reported directly to a user 113 without analysis or manipulation or with analysis or manipulation performed only locally, such as by the electronic control system 103.

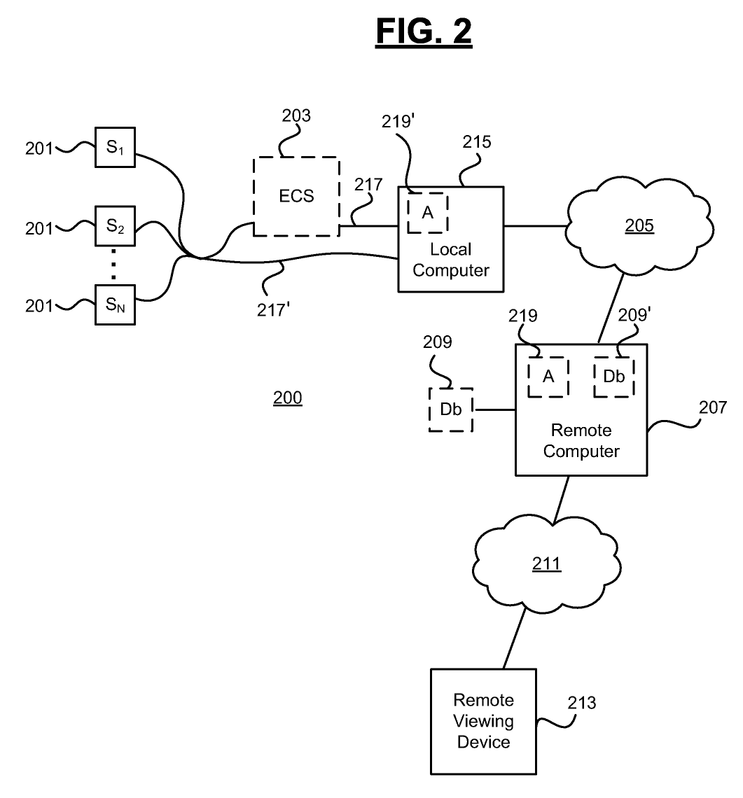

According to some embodiments as shown in FIG. 2, remote monitoring system 200 of the present invention may operate similarly to remote monitoring system 100 shown in FIG. 1 but further include a local computer 215, which may locally store, process, access, analyze, and/or manipulate raw data obtained from one or more sensors 201 of the water treatment system before being transmitted by a mode of transmission 205 to remote computer 207. Other aspects of these embodiments may be similar or identical to those described above in relation to FIG. 1. Remote monitoring system 200 may optionally include an electronic control system 203 linked to sensors 201, and local computer 215 may access capture, or receive data from one or more sensors 201 via an electronic control system 203 using a local connection 217, and/or directly from sensors 201 via local connection 217′ especially in the absence of an electronic control system 203. Local computer 215 may then transmit data by any suitable mode of transmission 205 to remote computer 207, and data may be stored in a remote database 209 associated with remote computer 207. Alternatively, a remote database 209′ may be located on remote computer 207 for storing the data. Following analysis or manipulation by an analyzer 219, 219′, the information, data, and/or analysis result(s) or report(s) may then be sent as an output to a remote viewing device 213 for viewing by a user using any suitable mode of communication 211.

According to some embodiments, the analyzer 219 may be located on or executed by the remote computer 207. Alternatively, the analyzer 219, 219′ may be located on or executed by the remote computer 207 and/or the local computer 215. According to embodiments having an analyzer 219′ located on or executed by local computer 215, the local computer 215 may send observational data in addition to other information of data to remote computer 207 via a mode of transmission. Such observational data may be data or information derived or synthesized from raw data obtained from the one or more sensors 201 that has been analyzed or manipulated by analyzer 219′. Data transmitted from local computer 215 to remote computer 207 may include data and information, such as an analysis result(s) or analysis report(s), relating to the qualities, characteristics, properties, etc., of the water being treated and/or the operation of the water treatment system.

According to embodiments of the present invention, local computer may be any type of computer, processor, or device able to (1) at least temporarily store, assemble, collect, aggregate, etc., data from one or more sensors, and (2) transmit data or information to a remote computer (or a remote database associated with the remote computer) via a mode of transmission. Thus, a local computer may contain or include (1) a memory device(s) to store, assemble, collect, aggregate, etc., the data at least temporarily, (2) one or more ports or inputs for receiving data or information either directly or indirectly from one or more sensors, and (3) a transmission interface(s) to transmit data or information to a remote computer. Such a local computer may further have the ability to process, manipulate, analyze, etc., the data obtained from the one or more sensors, such as by an analyzer or software located on local computer, prior to transmission of data or information to the remote computer and/or remote database. The data sent from the local computer to the remote computer and/or remote database may be observational data synthesized from data derived from one or more sensors. The local computer may be located at or near a water treatment system and/or the site(s) of one or more sensors within a water treatment system which may include a distribution system or collection system. The remote monitoring system of the present invention may comprise one or more local computers each associated with one or more sensors to collect, store, and/or transmit data or information derived from the one or more sensors to a remote computer via a mode of transmission. Each of the one or more local computers may transmit the data or information to the remote computer via the same or different mode(s) of transmission.

According to some embodiments, local computer may comprise a logger device located at or near site(s) of at least one sensor. Such a logger device may include one or more sensor ports for receiving data through cables, wires, etc., from one or more sensors. Alternatively, such a logger device may be capable of receiving data wirelessly from one or more sensors. To store or log (at least temporarily) data or information received ultimately from the one or more sensors and/or manipulated or analyzed, logger device may have any type of memory device known in the art, such as a drive, flash or SIM card, etc. Thus, logger device may further include an analyzer or software to analyze or manipulate the data from the one or more sensors. The logger device may have a transmission interface, such as wireless connectivity or antenna or other connection outputs, for communicating via a mode of transmission to a remote computer or server.

According to some embodiments, the logger device may have inputs, connectors, or ports for a plurality of sensors, such as at least four sensors, which may be automatically detected for plug-and-play options. The logger device may be able to store or log data for a greater number of values or measurements than ports, such as up to 16 values. Each sensor port may receive data from a sensor comprised of multiple individual sensors. The logger device may have different power options, such as battery power, auxiliary (external) battery power, reusable source (e.g., solar panel, etc.), and/or power from the electrical grid which may be combined with power switching (i.e., using battery or auxiliary power as a back-up). The logger device may further have inputs, connectors, or ports for receiving auxiliary power or a data communication link for connecting to a user computer or laptop. The logger device may also have a user interface for providing basic indications/information, such as device or sensor status, connections, etc. The logger device may be water-tight, enclosed, and/or have a rugged construction, may contain a desiccant to control moisture within the device, and/or may include a means for mounting the device. An example of a flow logger may include any FLO-LOGGER® product known in the art.

According to embodiments of the present invention, raw data about the operation of a water treatment system or the characteristics, conditions, qualities, properties, etc., of water processed or treated by a water treatment system may be acquired, collected, detected, measured, etc., by one or more sensors or probes placed at one or more sites or locations within or throughout the water treatment system, such as a plurality of locations within or throughout the water treatment system, which may include sites in the field, i.e., in a collection or distribution system. Sensors may be strategically placed to gather relevant data and information at appropriate sites or locations and/or provide logical functional groupings for review and analysis.

According to embodiments of the present invention, the one or more sensors may be used to obtain relevant raw data about the operation of a water treatment system and/or the quality of water being processed, treated, received, distributed, etc., that would be relevant to the analysis, manipulation, and evaluation of the data in generating an output, such as an analysis result, analysis report, alarm, etc. For example, each of the one or more sensors may be used to measure, quantify, or detect the following characteristics, conditions, qualities, properties, etc., of water. Examples of characteristics, conditions, qualities, properties, etc., of water that may be measured by the one or more sensors may include, but are not limited to: water temperature, chemical composition including total organic carbon (TOC), total suspended particles, quantity, flow rate, and types and amounts of waste(s) such as those commonly discharged into streams from waste water treatment or industrial sites. Further examples of characteristics, conditions, qualities, properties, etc., of water that may be measured by the one or more sensors may include contaminant(s), conductivity, pH, pressure, turbidity, permeate flow, dissolved oxygen, chlorine or fluorine concentration(s), tank or water level(s), and equipment status and operation. According to some embodiments, the one or more sensors may be chosen to generate data or information for a regulatory report necessary to enable a regulatory agency to determine operational parameters and quality and quantity of the treated water such as water production rate (flow), treated water consumption rate (flow), treated water storage volume, reserve capacity (at current production and consumption rates), final treated water quality, reports and archive data for regulatory compliance and/or QA/QC documentation. According to embodiments of the present invention, examples of sensors that may be used with the remote monitoring system of the present invention may include any sensor known or used in the art. In addition to the variables listed above, the one or more sensors may be used to measure water level and/or flow velocity using any technology either known or later developed in the art. Such measurements may, for example, be used in combination to determine volumetric flow rate along with other known conditions and constants. An example of a sensor may further include a rain gauge. Examples of flow velocity or area flow velocity sensors that may be used with embodiments of the present invention may include wafer sensors and any sensor based on Doppler or ultrasonic, radar, pressure flow, electromagnetic (EM), magnetic (e.g., surcharge), etc., technology or detection. Examples of level, height, or depth sensors that may be used with embodiments of the present invention may include any based on ultrasonic (look-down, submerged look-up, in-pipe, etc.), pressure (e.g., bubbler, surcharge, diaphragm displacement, etc.), radar, etc., technology or detection. According to some embodiments, a height or level sensor may be combined with other structural elements or devices, such as flumes and weirs, to deduce other measurements or states, such as velocity in addition to water level, based on known relationships and constants. According to some embodiments, any of the one or more sensors may further include an internal or external temperature sensor to provide, for example, auto correction for effects of temperature on any primary measurement by the sensor. A sensor according to some embodiments of the present invention may each comprise a plurality of sensors, which may then be jointly fed into a local computer, such as a logger device.

According to embodiments of the present invention, the one or more sensors may include any products on the market, sold, made by, or branded under, for example, Hach™ Sigma™ or American Sigma™, Marsh-McBirney™, etc., either known or later developed in the art. Particular examples of the one or more sensors may include FLO-DAR®, FLO-TOTE®, FLO-MATE®, etc., sensors. For additional description of some types of sensors, see, e.g., U.S. Pat. Nos. 5,506,791, 5,633,809, 5,691,914, 6,208,943, 5,644,088, 5,811,688, 5,544,531, and 5,315,880, the contents and disclosures of which are hereby incorporated by reference in their entirety.

In the case of water districts, electronic sensors may be used to detect or measure the amount of storage, discharge pressure and flow from the systems. Other parameters may be determined by analytical tests. Many of the sensors used to continuously monitor water treatment operations are based on advanced separation processes employing selective ion membranes which concentrate the analyte for detection. For example, detection of chlorine may be mediated via an ion selective membrane which may readily and specifically pass an analyte, such as free chlorine or hypochlorous acid (HOCl), thus separating and concentrating the analyte from the bulk solution. The sensors may incorporate multiple sensors as part of a single detector unit.

The presence or absence of turbidity in the water supply may greatly affect the amount of disinfectant required to achieve inactivation of biological organisms. The suspended particles producing turbidity are usually removed in the water treatment process before disinfection agents are applied. However, turbidity breakthroughs do occur and failure to quickly raise the disinfection dose level may lead to insufficient disinfection residuals reaching the distribution system. This may present a threat to public health, particularly if the drinking water supply is contaminated either deliberately or inadvertently.

According to embodiments of the present invention, the one or more sensors may optionally be integrated with or connected to an electronic control system. The electronic control system may generally be used to control the operation of a water treatment system by local operators. Examples of an electronic control system may include an in-house Supervisory Control and Data Acquisition System (SCADA) or a Progammable Logic Controller (PLC). The electronic control system may be composed of any available commercial devices for converting analog to digital, such as Analog to Digital boards, specifically designed for the purpose of converting instrument readings or data to computer readable form. Thus, the remote monitoring system of the present invention may utilize existing instrumentation and control systems as well as existing communication devices. The electronic control system may perform basic analysis of the raw data to produce an analysis parameter that may then be sent to the remote computer. According to some embodiments, the electronic control system may continuously scan the sensor data and automatically log and archive the data at specified intervals. According to some embodiments, raw data obtained from a sensor may be stamped or labeled with time and location information, such as a unique identifier(s), for aiding subsequent analysis or manipulation. Raw data obtained from a sensor may also be labeled according to the particular order in which the data is sent to a remote computer. According to some embodiments, the electronic control system may include a transmission interface which functions to transmit the data to the remote computer.

According to some embodiments, the remote monitoring system may further include a local computer located at or near the physical location of the water treatment system and/or the site(s) of one or more sensors within a water treatment system which may include a distribution system or collection system. For example, the local computer may be a logger device as described above. The local computer may read, query, access the data collected from the one or more sensors of the water treatment system, store in an appropriate electronic format at least transiently, process, manipulate, analyze, etc., the data obtained from the one or more sensors, such as by an analyzer or software located on local computer, and/or transmit the data to the remote computer. For example, storage of the data on the local computer may provide an on-site data backup, and the data may be added to an historical data file for use in analysis to allow a current data file to be reused for new data collection. According to some embodiments, the local computer may be connected to the electronic control system and access the data via the electronic control system. Any type of connection, electronic or otherwise, may be used, such as, for example, a serial interface board, a USB interface card, a network connection, wiring, etc. According to some embodiments, a user may use the local computer to view or display the data or results or reports generated from the data stored and/or analyzed, manipulated, etc. on a remote computer.

According to some embodiments, a local configuration file on the local computer may tell a program on the local computer which of the register addresses of the electronic control system to access, any scaling factor which needs to be applied, a physical description of the data being collected, etc. The data set collected may then be converted into a form for transmission, such as a comma delimited string value, and perhaps stored locally and possibly encrypted for security on a storage medium such as a hard disk, etc.

According to embodiments of the present invention, the data and information obtained, acquired, collected, detected, measured, etc., from the one or more sensors may be transmitted to a remote computer, located off-site, using any known or available mode of transmission. The data and information may be transmitted either directly from the one or more sensors, from the electronic control system, or from a local computer connected to the electronic control system and/or directly to the one or more sensors. Once transmitted and received by the remote computer, the data and information may then be remotely stored on the remote computer and/or a remote database on or associated with the remote computer. According to some embodiments, the data and information may then be manipulated on the remote computer to generate an output, such as an analysis result, report, alarm, etc., that may be communicated to a user, and/or the data and information used to generate an output may be manipulated on the local computer prior to transmission to the remote computer. Such data or information transmitted from a local computer may include observational data which is calculated, manipulated, etc., by an analyzer on the local computer from data derived from one or more sensors. According to some embodiments, the data and information may be analyzed, manipulated, etc., by analyzer(s) located on both the remote computer and the local computer.

According to embodiments of the present invention, the remote monitoring system of the present invention may further comprise a remote database or software-implemented remote database associated with the remote computer for storage of data. The remote database may be on the remote computer or exist as a separate unit, and the number of remote computer(s) and/or remote database(s) may be varied to suit a particular application, network traffic, or demands of a particular client. According to some embodiments, for example, the remote computer may comprise a computer, an ftp server, a remote database, and/or a web or internet server, which may each be located at the same or different locations and use any available and appropriate operating systems. This storage on the remote database may take many forms such as flat files, spreadsheets, and relational or non-relational databases. According to some embodiments, for example, the remote database may be a relational database, such as Microsoft SQL Server or Oracle database products.

According to embodiments of the present invention, the exact mode of transmission may vary depending on the circumstances. Any suitable technology or device known and available in the art for transmitting data to a remote or physically separated computer is contemplated for use as a mode of transmission according to embodiments of the present invention. Examples of modes of transmission may be achieved through any suitable medium. According to embodiments of the present invention, the data may be transmitted, for example, continuously, in real time, at periodic or selected intervals, on condition, or on demand by a user. The data may also be encrypted for security for additional security, and may be decoded by the remote computer and/or the remote database and placed in the appropriate locations.

According to some embodiments, the data may be transmitted to the remote computer directly by sensor assemblies comprising the one or more sensors. According to these embodiments, the one or more sensors may be fitted with communications processors which enable the sensors to send data directly to the remote computer. Suitable instruments may include sensor assemblies having a transmission interface effective for real time data transmission, such as a LonWorks®.RTM network variable interface. Suitable sensors may also include, for example, the Six-CENSE®.TM and the CT-CENSE®.TM manufactured by Dascore, Inc., as well as the multi-sensor devices manufactured by Sensicore, Inc. In this example, sensors may transmit the data to a remote computer by any suitable mode of transmission known in the art, such as an Internet server computer, and may be connected to a remote computer through existing telephone wiring on a dedicated network connection or cell network.

According to some embodiments, the data may be transmitted to the remote computer via an electronic control system connected or coupled to the one or more sensors using any suitable mode of transmission known in the art. For example, a section of ladder logic or function block program code may be inserted into the code base of the electronic control system which directs the electronic control system to send specified data to the remote computer and/or database. The communications protocol may be any protocol supported by the electronic control system which facilitates the transmission. For example, RSLinx®, a software program from Rockwell Software, may be operative on the remote database computer to facilitate the transmission by a PLC. Alternatively, any number of commercial communications drivers may be used such as those produced by commercial providers such as Kepware®, Wonderware®, and so on. In the case of an electronic control system typified by SCADA® or HMI® products, such as Wonderware®, RSView®, WinCC®, and other similar products, code blocks may be added to the control code to allow the operating program to collect and send data to the remote computer. Thus, the steps of collecting data locally, possibly storing it temporarily, and subsequently transmitting this data to a remote computer may be incorporated into the electronic control system.

According to some embodiments, the data may be transmitted to the remote computer via a local computer connected or coupled to the one or more sensors directly or through an electronic control system connected or coupled to the one or more sensors. According to these embodiments, the local computer may transmit the data acquired or collected directly or indirectly from the one or more sensors to the remote computer by any suitable mode of transmission known in the art. According to some embodiments, for example, the local computer may comprise a logger device as described above located at or near site(s) of at least one sensor.

According to embodiments of the present invention, after the data and information obtained from the one or more sensors has been sent to the remote computer of the remote monitoring system, the remote computer may analyze or manipulate the data to generate an output, such as manipulated data, an analysis result, an analysis report, an alarm, etc. Alternatively, the local computer may analyze or manipulate the data and information obtained from the one or more sensors which may then be transmitted to the remote computer, and the remote computer may then further analyze or manipulate the data and information to generate an output. However, the output may be generated, presented, uploaded, etc., by the remote computer without further analysis or manipulation by the remote computer. The analysis, manipulation, etc., of the data may be performed by an analyzer, such as a software program or routine, firmware, and/or hardware, that may be housed on the local computer, the remote computer, and/or the remote database associated with the remote computer.

According to embodiments of the present invention, the analyzer may be one or more software program(s) on the remote computer and/or on the local computer. Such an analyzer may perform analysis, calculation, comparison, manipulation, etc., of the data to generate an output, such as an analysis result, an analysis report, an alarm, etc., relevant to the monitoring of a water treatment system, and the analysis, calculation, comparison, manipulation, etc., may be performed continuously, in real time, at periodic or selected intervals, on condition, or on demand. According to embodiments of the present invention, an analyzer may be used to make calculations based on a combination of raw data from multiple sensors. When the analyzer is located on a local computer, the analyzer may be used to generate or synthesize observational data derived from raw data obtained from a plurality of sensors. For example, independent data measurements of (1) flow rate and (2) water level by multiple sensors may be combined and used to calculate volumetric flow (in units of volume per time) based on the known dimensions and other constants regarding a water channel, pipe, etc., at a site within a water treatment system. Such multiple sensors used to measure volumetric flow may be connected to a common local computer, such as a logger device.

According to embodiments of the present invention, the data acquired or collected from the one or more sensors may be compared by the analyzer to expected or historical performance data or records and/or to any known values and constants, such as known or expected transit times, location-specific flow rates and patterns, and distances within different portions of a water treatment system, known physical and chemical properties and characteristics of water, contaminants, disinfectants, pollutants, etc., using any known equations, algorithms, etc., which may be used to model, predict, or compare the performance of the water treatment system or the quality of water processed or treated by the water treatment system. Data acquired or collected from the one or more sensors may be compared to each other and/or to historical data, and calculations may be performed to generate an output, such as an analysis result(s), etc. According to embodiments of the present invention, the analyzer or software may perform any calculation, computation, comparison, analysis, etc., that would be relevant, suitable, or appropriate to monitoring of the operation of a water treatment system or the processing or treatment of water in a water treatment system.

According to some embodiments, an analyzer on the local computer, the remote computer, and/or remote database associated with the remote computer may also interpret and consider any identifier(s) or configuration files associated with the data that may indicate or identify the origin, location, and time of the data capture from the one or more sensors. The analysis and calculation of the data may further be performed by the analyzer to determine or indicate performance, evaluation, preventative maintenance, scheduling, optimization, and trouble shooting of the operation of the water treatment system or equipment, in addition to monitoring water quality. For example, the data may be compared to known or expected performance data or parameters to calculate a differential, which may be used to determine if the water treatment system is performing within a normal range or out of bounds if a predetermined differential is exceeded. Such comparisons may be based on the amount or concentration of, for example, a disinfectant, contaminant, or pollutant present at different locations in a water treatment system. If the differential is exceeded, then appropriate persons, operators, and/or agencies may be alerted. Alternatively, for example, the data may be compared to known, expected, or historical data or values to determine if the operation of the water treatment system is optimized.

According to some embodiments, the analyzer may convert the data into a consistent set of units, and thus translates all values into a common format, such as pounds per square inch (psi) for pressure, etc., using a units conversion sub-program to allow for appropriate comparisons and calculations. Furthermore, the data may be normalized to specific configurations and conditions for a water treatment system. For example, the feed pressure may be critical in determining the future and current performance of a system in reference to its performance when new. For reverse osmosis membranes, changes in pressure are related to age, production rate, and temperature and vice versa. Thus, a change in flow rate may or may not indicate that the overall system's performance has changed when normalized and compared to its performance when new or recently cleaned. Prior to this invention, the complex mathematics for these conversions required some manual intervention on the part of the operator to compute the normalized conditions. Embodiments of the instant invention may do this automatically and report normalized data to the output.

According to some embodiments, the analyzer or software of the present remote monitoring system may be used to make any suitable statistical inferences, derivations, conclusions, or predictions from the data, especially based on a comparison to historical data or expected values. Such an analysis or manipulation of the data may provide an indicator of either normal or abnormal operation of a water treatment system or characteristics, properties, qualities, etc. of water processed or treated by a water treatment system. According to some embodiments, the analyzer may be used to predict conditions, such as the presence, quantity, or concentration of a disinfectant, contaminant, or pollutant at a downstream location at a later point in time based on data obtained from sensors at upstream locations within a water treatment system.

For example, in the context of a water treatment facility for providing potable drinking water to the public, data, disinfectant concentration and turbidity, may be analyzed from both the treatment facility and the distribution system, and historical information as well as known constants may be used to predict expected conditions at points downstream within the distribution system based on expected lag times and the effluent conditions from the treatment facility. For example, data may be collected from the water treatment facility about relevant information, such as chemical dosing rates, filtered water turbidity, chlorine residual, etc. as well as data from sensors in the distribution system, such as chlorine residual, etc., may be used for comparison. With historical data as a reference point, one can calculate a chlorine demand from the chemical dose rates, flows, and residual using the current data. Chlorine Demand may be defined as the actual amount of chlorine which is reacting, typically calculated as free chlorine dosed less the residual. Chlorine demand may be correlated with temperature, season, and filtered water turbidity. Additionally, residual chlorine leaving the plant may be correlated with residual chlorine within the distribution system. If the actual chlorine residual measured at the distribution system point of measurement varies from the historical values expected from the chlorine residual leaving the treatment facility by more than a set percentage or number of standard deviations, then an alarm or alert may be issued by the remote monitoring system of the instant invention.

As another example in the context of a water treatment facility for providing potable drinking water to the public, data obtained from the one or more sensors may be combined with known system constants such as flow rates, residence times, and so on, to continuously generate a calculated product of disinfectant concentration times contact time C*T. This simple factor alone is quite useful in predicting the amount of biological organism deactivation.

As another example in the context of a waste water treatment plant (WWTP), an analysis or manipulation of data obtained from sensors at upstream locations in a collection system, such as sites or locations of discharge from water treatment or industrial waste water plants, to detect the amount of a contaminant, pollutant, may be used to predict the future composition and flow rate of water arriving at the central WWTP. This may be accomplished in a simple manner by using known or expected constants and information as well as historical records about transit time, flow rates and patterns, etc., from each of the relevant sites or locations upstream, such as within the collection system and at or near points of discharge. Any results, conclusions, reports, etc., generated using such an analysis or manipulation may be used to alert operators of a central WWTP receiving waste water from the collection system of a potential overload so that appropriate precautions and changes in operation may be made. As will be readily appreciated by those skilled in the art of data analysis, this can provide a powerful indicator of either normal conditions expected at the WWTP or out of bounds conditions that may require immediate action and notification of responsible parties.

According to other embodiments, the projected or remaining life of equipment, such as a membrane, may be determined or estimated by the remote monitoring system based on operational performance data. Efficiency levels for equipment or a water treatment system as a whole may be determined by the remote monitoring system relative to a theoretical potential or efficiency, which may be based on a theoretical minimum water, power, and chemical consumption versus actual consumption calculated. In addition, financial and economic reports may also be generated based on performance and/or consumption data. Furthermore, the data may be analyzed and compared to federal and/or state regulatory requirements for water quality and environmental protections.

According to some embodiments, the information and data may be displayed or presented as an output, such as an analysis result(s) and/or analysis report(s), in a predetermined format, which may then be sent to a user, such as, for example, a consumer, public official, authorized personnel, or regulatory agency. Indeed, the data may be manipulated and formatted into an output or analysis report as required for submission to a regulatory agency. According to some embodiments, the analysis or manipulation of data may be presented as an output that is uploaded onto to a web server and made accessible via a web browser for presentation to, for example, a public official, consumer, or interested member of the public. Alternatively, according to some embodiments, an output in the form of an alarm may be sent to alert a user of a problem or deviation from normal conditions.

According to embodiments of the present invention, once the data is analyzed or manipulated into an output, such as an analysis result or analysis report, the output may be sent by any known, available, and/or suitable mode of communication from the remote computer to a remote viewing device for viewing by a user. According to some embodiments, the output may be sent to the remote viewing device or accessed by the remote viewing device continuously, in real time, at periodic or selected intervals, on condition, or on demand. For example, the output may be a notification, alarm, or alert, such as an Alarm Event, sent on condition of an emergency or abnormal, harmful, or dangerous quality, state, or condition relating to a water treatment system. Such an output may include a notification of failures, shutdowns, exceeding of critical parameters, equipment damage, etc. Alternatively, for example, the output may be composed as an analysis report, which may be in a format for submission to a regulatory and/or law enforcement agency. The remote monitoring system may send, present, or upload an output as a weekly, monthly, yearly, etc. summary of performance, water quality, or other information that may be reviewed by management for the water treatment system or by elected officials, customers, vendors, or members of the public. Alternatively, the remote monitoring system may send, present, or upload an output continuously, on condition, or on demand of a user. When sent or presented, the output may reflect or show updated information and recently collected data.

According to some embodiments, the format and sophistication of the presentation of the output will likely depend on the intended recipient(s) or user(s). For example, an output, which may include any relevant information, data, analysis, results, reports, etc., about the operation of a water treatment system or the quality, properties, etc., of water processed or treated by the water treatment system, may be presented in a more sophisticated form when presented to internal management or operators of the water treatment system than when presented to elected officials, customers, or members of the public.

According to embodiments of the present invention, one or more output(s) may be sent, presented, or uploaded to one or more remote viewing device(s) in one or more formats having different sophistication or complexity based on their intended recipient(s) or user(s), even if such one or more output(s) relates to the same data or information. According to some embodiments, an output, such as an analysis result or analysis report about current data may be presented alongside and/or in comparison to historical records. An output may also be used to present scheduled and predicted maintenance reports. For example, the output may provide or present preconfigured performance information, maintenance, quality assurance, quality control, regulatory, cost reports, performance evaluation, graphing, historical trends, regulatory reports plant or facility process, operating and economic information, indications and scheduling for preventative maintenance, troubleshooting, etc. According to some embodiments, access to an output of the present remote monitoring system may depend on the security measures in place, such as a login and password or other identifying criteria.

According to some embodiments, the output may be used to report or present information or analysis of the operation or conditions in a waste water treatment plant (WWTP) particularly as it relates to health and safety concerns. The analysis result may take many different forms; however, one form may be a prediction of the water composition and flow rate in terms of selected parameters of interest that may arrive at a WWTP as a function of time. Thus, for example, the remote computer may be operable to calculate a predicted concentration of various components at the time of their arrival at a central WWTP and compare the computed values with pre-established and/or historical parameters.

According to some embodiments, the output may be a report submitted to a regulatory agency in a required format, such as visual graphs, statistical reports, or a compliance calendar, to meet the reporting requirements of the agency, and such reporting or sending of the output may be performed automatically. Quality and safety standards for potable water are regulated by the Environmental Protection Agency (EPA) in accordance with the Public Water System Supervision program. The standards are enforced by local agencies. There are over 170,000 water districts in the United States which provide public drinking water to 90% of Americans. The EPA has primary standards designed to protect public health against substances that may be harmful to humans if consumed. EPA secondary standards ensure that aesthetic qualities of water, such as taste, odor, or clarity, are met. However, each water district remains responsible for monitoring the drinking water itself to ensure that it meets all drinking water standards. The treatment processes for the drinking water must be monitored as well. Therefore, the remote monitoring system of the present invention may be useful in not only monitoring whether these standards are met on a routine and continuous basis, but also providing automatic generation of regulatory reports as an output to an agency in the required format.

According to some embodiments, the remote monitoring system of the present invention may automatically prepare the documentation required to meet the regulatory requirements. Such documentation may be printed out and mailed or transmitted by a suitable mode of communication, such as by facsimile, ftp, or email, to the regulatory agency, thereby reducing or eliminating the opportunity for human error and/or unwanted manipulation. In order to comply with the regulatory testing calendar, water districts are generally required to report a list of analytical test results varying from hourly to yearly, depending on the source of the water supply. Monitoring schedules may differ according to the type of contaminants that may be present in a given water supply. The hourly tests may typically include chlorine and turbidity, which may be measured or collected automatically.

According to some embodiments, the output of the remote monitoring system may be a regulatory report sent to the department of Homeland Security and/or law enforcement agencies in situations appearing to suggest deliberate tampering of a water treatment system, such as by an act of terrorism. Embodiments of the present invention may be able to carry out sophisticated calculations, manipulations, analysis, etc. to detect tampering events and perhaps distinguish those events from normal malfunction or mismanagement.

According to embodiments of the present invention, the output may be in any format and may incorporate a tabular or graphical display as may be suitable to facilitate or focus the presentation of the data or analysis or manipulation of the data for a particular user(s). According to some embodiments, the output of the remote monitoring system may be a simplified presentation for a non-technical user that is untrained or lacks detailed knowledge about the operation of a water treatment system, such as a customer, elected official, or member of the public. For example, municipal water treatment plants are ultimately the responsibility of elected officials. Yet these officials rarely have the technical training or time to allow them directly access the performance parameters of the systems for which they are responsible. Embodiments of the present invention may easily be used to provide a readily understandable presentation output of the current performance of a municipal water treatment system. Such an output may be made accessible to the public, such as via the Internet by uploading onto a web page, thus allowing interested members of the public to monitor the operation of their own drinking water plants as desired. In providing a simplified presentation of the data to the non-technical user, operating parameters may be color coded and displayed graphically or in a tabular format, etc.

However, according to some embodiments, a simplified presentation of the data in an output of the remote monitoring system may be beneficial to even a trained operator or manager of a water treatment system. Accordingly, a graphical and/or color coded presentation of the data or analysis or manipulation of the data may potentially be used in any output format or report. A graphical presentation may include any suitable graphical format, such as tables, pie charts, bar graphs, etc., that may aid the presentation of the output or report. Color coding may be used, for example, to provide an indication of normal or abnormal operation, as well as warning status or alarm conditions. An output of the remote monitoring system may also show data or analysis or manipulation of the data in a geographical layout or form to help track or pinpoint the origin or cause of a problem. Historical data or expected values may also be shown with current data for comparison. When an output is provided to a trained user, such as a manager or operator of a water treatment system, the data and/or analysis may be presented as an exception report showing all instances where data triggered an alarm or were close to a trigger point.

According to embodiments where an output is sent or presented to management, the outputs or reports may be typically generated for three primary management levels: (A) Process systems operations, (B) Plant quality assurance (QA)/quality control (QC), and (C) financial oversight. For instance, an output or report for operations of a process system may contain information necessary to monitor, maintain, supervise, and trouble shoot process plant system performance. In this manner, typical information and parameters may include, if applicable, flow rates, pressures, delta pressures, permeate and/or ion exchange quality, pH, alarm conditions, tank levels, and a graphical presentation of applicable process performance parameters and trends.

A Plant QA/QC output or report, for example, may contain information necessary to enable plant managers to effectively manage downstream manufacturing or distribution processes. In addition, quality assurance personnel may be able to monitor the quality and quantity of the treated water to confirm compliance with specifications and standards. Information in this report may typically include treated water production rate (flow), treated water consumption rate (flow), treated water storage volume, reserve capacity (at current production and consumption rates), final treated water quality, reports and archive data for regulatory compliance and/or QA/QC documentation.

Financial oversight may be achieved with a plant economic output or report which may contain information needed by managers with profit and loss or budget responsibility to effectively track the cost of operation and to identify budget variances, when they occur, to permit timely corrective action. For this purpose, typical information parameters contained in a plant economic report may include calculated power consumption (expressed in kWh and actual cost in local currency) and computed on the basis of user's supply pump/motor efficiencies both as a year to date, as a percent of the prior period, and variances both actual and budget/actual versus prior period. The parameter may also include calculated chemical consumption (expressed in volume consumption and as converted to local currency) and computed based on the user's supplied chemical dose rates and integrated feed water flow rates. This may be performed as a year to date, as a percent of the prior period, or as variances both actual versus budget/actual versus prior period.

According to embodiments of the present invention, an output including data, analysis, results, analysis reports, etc., may be sent to a remote viewing device using any appropriate or suitable mode of communication known in the art. The output may be in any suitable file format, such as but not limited to: html, jpeg, gif, pdf, etc., based on the output type and/or remote viewing device. The output may be sent in a suitable and/or tailored format to preselected recipients, such as authorized personnel or operators of a water treatment system, law enforcement, and/or regulatory agencies, in the event of an emergency or abnormal conditions or operation. The content of the output may be kept confidential, and access to the output including data, analysis, results, analysis reports, etc., may be controlled by encryption or the use of appropriate account names, protocols and passwords. Multiple parties or persons may be notified, access, or receive outputs from the remote monitoring system, thus allowing redundancy in sending notifications, alarms, analysis results, analysis reports, etc.

According to some embodiments, the mode of communication for sending an output to, or allowing access to an output by, a remote viewing device may vary and may use any suitable technology. For example, according to some embodiments, an output including data, analysis results, analysis reports, etc., may be uploaded to an Internet or web server for access, visualization, or downloading by a remote viewing device, such as by using a web browser. According to some embodiments, the Internet or web server may be the remote computer of the remote monitoring system or a separate computer or server. According to some embodiments, the output may be uploaded to an Internet or web server for access with little or no manipulation or analysis by the remote computer, visualization, or downloading by a remote viewing device by a user. According to these embodiments, for example, the data or information derived from the one or more sensors may first be analyzed or manipulated by the local computer prior to being transmitted to the remote computer. By making the output available on an Internet web server, the communication or dispersion of the output, including data, analysis results, analysis reports, alerts, alarms, etc., may be greatly facilitated and may involve any interested or authorized recipients. For example, any authorized recipients may access data, analysis results, analysis reports, alerts, alarms, etc., of the output on a webpage by accessing the data, information, output, etc. asynchronously from the Internet server computer. Furthermore, the output, including data, analysis, results, analysis reports, alerts, alarms, etc., may be continuously or regularly updated and made available in near real time.

According to some embodiments, the mode of communication for sending an output to, or allowing access to an output by, a remote viewing device may include other suitable technologies, such as, for example, by facsimile, file transfer protocol (FTP), voice or text messaging, text to voice telephone messages, electronic mail, pager, human voice calling, SMS messages, instant messaging or groupware protocols, or other messaging medium which can be mediated by a computer program connected to a phone line, public switched telephone network (e.g. via telefax), the Internet, a cellular network, wireless or satellite communication, radio communication, etc. See description above for additional examples of a mode of communication. Examples of remote viewing devices that may be used with embodiments of the present invention may include, for example, personal computers, servers, etc., as well as a variety of personal communications equipment, such as PDAs, cell phones, pagers, Blackberrys®, Palm® devices, iPhones®, etc. According to some embodiments, the remote viewing device may be the same as the remote computer of the present remote monitoring system.

According to embodiments of the present invention, remote computer 107, 207 of remote monitoring system 100, 200 in reference to FIGS. 1 and 2 is located at a different and physically distinct and remote location than the water treatment system, which may include local computer 215. The remote computer 107, 207 of remote monitoring system 100, 200 may not be used to remotely control or direct controls for a water treatment system, such as an electronic control system 103, 203. Indeed, according to embodiments of the present invention, the only communicative or electronic link or connection between (1) the remote computer and (2) the water treatment system or the sensors, electronic control system, and/or local computer located within the water treatment system may be the mode of transmission of the present remote monitoring system. Several benefits and advantages may be achieved by physically separating the storage, manipulation, analysis, reporting, etc., functions of the remote computer and/or remote database of the present invention from the site(s) or location(s) of data collection (i.e., sensors) within a water treatment system, which may further include a broader distribution or collection system.

One advantage of embodiments of the present invention, for example, is that remote storage and manipulation of water quality and treatment data may make the operation of a water treatment system safer and less susceptible to tampering or control by unauthorized individuals or outsiders by separating the operation and control of the water treatment system from the data analysis, manipulation, and/or communicating or reporting functions of the present invention. For example, this feature may be useful in detecting direct tampering, such as an act of terrorism, by an individual or outsider on a water treatment system. According to embodiments of the present invention, since the remote computer of the remote monitoring system is physically separated from the operation of the water treatment system, it is unlikely that an individual tampering with a water treatment system would also have access to the remote monitoring system of the present invention, especially since access to the remote monitoring system may be controlled or password protected. According to these embodiments, if a hacker were to remotely access the remote monitoring system of the present invention, they would not be able to directly access and control the operation of the water treatment system because the remote computer and database is external, physically remote, and not connected to the process facility being monitored except perhaps via a mode of transmission.

Another advantage of embodiments of the present invention, for example, is that the ability to send an output or other data, information, etc., about the operation of a water treatment system to a remote viewing device via a mode of communication may reduce the need for operators or authorized personnel to visit the sites of the water treatment system being monitored, maintained, etc. This may reduce the costs associated with monitoring a water treatment system if data had to be collected locally or by direct connection to a device or local computer. This is especially true if the remote monitoring system is further combined with sensors and other devices that require less maintenance and service, such as sensors that do not contact the water and are able to operate reliably for longer periods of time without maintenance or service.

Another advantage of embodiments of the present invention is that the remote monitoring system of the present invention may create a layer of redundancy that may be independent of and/or complementary to the direct monitoring carried out by qualified individuals at a water treatment system or facility to safeguard operation of the water treatment system. Redundancy may also be achieved by, perhaps simultaneously, reporting analyzed or manipulated data to multiple persons and/or entities in the same or different format(s). In addition, the remote monitoring system may reduce or eliminate the need for direct human involvement. By having the remote monitoring system automatically perform the calculations and manipulations on the raw data in real time without direct human involvement, there may be less human error in evaluating, analyzing, etc., water quality and the operation of the water treatment system.

Yet another advantage of embodiments of the present invention is that data and information may be combined, pooled, compiled, etc., from sensors placed at multiple location(s) or site(s) throughout a water treatment system and in the field as part of a broader distribution or collection system. According to some embodiments, sites or locations within the distribution or collection system may be considered part of the water treatment system even though the distribution or collection system may operate independently of a water treatment core facility of the water treatment system. Such sensors located at the multiple location(s) or site(s) may operate independently and/or have no communication between sensors other than the remote monitoring system of the present invention. By comparing data from these multiple independent sites or locations, a more advanced form of analysis and conclusions may be performed or made in view of the water treatment and distribution systems as a whole. For example, better prediction and anticipation of downstream contamination events may be made by having multiple data points obtained from sites or locations throughout a collection or distribution system associated with the water treatment system, thus allowing appropriate actions to be taken downstream to lessen or prevent the impact or damage caused by the contamination event, such as the introduction of dangerous, poisonous or unhealthful contaminants into the environment or drinking water.

For example, the water treatment core facility may be a central wastewater treatment plant (WWTP) that receives waste released from multiple sources upstream that converge into a common collection system that feeds into the central WWTP. The collection system may serve numerous waste water treatment sites or industrial waste sites that feed into a central WWTP. According to embodiments of the present invention, multiple sensors may be placed throughout a collection system including the water treatment and industrial waste sites to monitor discharge into the common collection system. Water treatment sites may include cities, manufacturers, agricultural operations, etc., which treat waste water before it is discharged into the common collection system. For a WWTP operator, an accurate prediction of the composition of incoming waste water would be highly beneficial for the efficient operation of the WWTP facility.

According to embodiments of the present invention, the composition of influx water in a WWTP serving a geographically distributed waste water collection system may be estimated from measurements taken from sensors located upstream, such as at or near waste water treatment site(s) or industrial waste site(s) discharging into the common collection system. Since the water flow patterns, transit times, and the composition of water leaving each of the treatment or industrial sites within the waste water collection system may be known, the expected composition of influx water arriving at the WWTP can be calculated and reliably and quickly transmitted to the operators of the central WWTP and/or remotely to other entities or persons, such as through a remote viewing device. In addition to known information, the volumetric flow rate may be measured using the one or more sensors. This advance notice allows the WWTP to respond to varying contaminant or pollutant introductions in a far more effective manner than at present, where the first knowledge or information may come after the contaminants have already entered or even passed through the system. For WWTP entities that operate reclamation facilities downstream of the WWTP, this advance knowledge is even more valuable as it allows the reclamation facility to modify its operations as necessary to prevent damage to the process facilities. It will be readily appreciated by WWTP operators that knowledge of the incoming waste water composition would be of great benefit in assuring the continued operation of the central facility at top efficiency.

Another advantage of embodiments of the present invention is that the cause, scope, or location of a problem or source of contamination may be better determined, tracked or distinguished by having more independent data points of reference obtained from sensors at sites or locations throughout a water treatment system, such as sites or locations in a water treatment core facility as well as throughout a collection or distribution system, i.e., in the field. Such analysis or determinations may be aided by the existence of historical data and known information about the operation of the water treatment system in relation to its environment which may be used for comparison. For example, a chemically or biologically active agent may be deliberately injected into the distribution system at a point downstream of a potable drinking water treatment facility. A sophisticated terrorist might first inject a chlorine scavenger, such as sodium metabisulfite, into the distribution system to eliminate the residual chlorine normally present. At some point downstream of the metabisulfite injection point, the chemical or biological agent could be injected into the water without destruction by any residual disinfectant. Without a remote monitoring system in place with sensors in the distribution system, such contamination could go undetected for quite some time, allowing a thorough infiltration of a biological or chemical agent throughout the distribution system. By contrast, the remote monitoring system could detect that the residual chlorine at the sensor had diminished to zero and sound the alarm. Especially with historical data available for comparison, the remote monitoring system would be able to reduce the incidence of false terrorist attack alarms because data obtained from sensors at the treatment facility and in the distribution system could be compared. For example, a chlorine-dosing equipment failure might be determined and distinguished from a terrorist attack if a fall in chlorine concentration is observed at both the water treatment plant and at points in the distribution system.

Another possible advantage of embodiments of the present invention is that the data may be transmitted to a remote computer where more advanced computations, manipulations, analysis, etc., may be performed prior to reporting, uploading, etc., of an output, such as an analysis result, analysis report, or alarm to a user. A software program on the remote computer may be more sophisticated than may be achieved locally, such as with the local electronic control systems used to control and operate the water treatment system, plant, or facility. This may allow for the processing power of existing control systems to not be impaired or impacted. For example, an analysis report generated by manipulation of the data on a remote computer may include a submission to a regulatory agency to meet reporting requirements in the format required by the agency, and such reporting may be performed automatically. The remote analysis, manipulation, etc., may be performed quickly and automatically to remotely monitor operation and water conditions in real time, continuously, at selected, periodic, or regular intervals, on condition, or upon demand of a user and rapidly generate multiple types of outputs, such as alarms, analysis results, analysis reports, etc., to one or more users. For example, the software program may separately generate a detailed regulatory report for submission to a regulatory agency, send a simple alarm to authorized personnel to alert of a contamination or equipment failure, and/or post data and information about the water treatment system on a web page for access by a member of a public. Alternatively, the analysis, manipulation, etc., of data and information may be performed locally on the local computer, such as a logger device. According to some embodiments, such analysis, manipulation, etc., of data and information on the local computer may be performed in addition to further analysis, manipulation, etc., of data and information on the remote computer.

Yet another advantage of embodiments of the present invention is that greater flexibility and accessibility may be achieved over existing systems allowing access to the remote computer to receive data, information, reports, etc., sent by any known means or mode of communication from the remote computer. By having greater accessibility and communication of data, information, reports, etc., greater coordination may be achieved between different parts of the water treatment system and any associated collection or distribution system, which may include, for example, remote sites or locations of industrial waste discharge in the case of a WWTP.

Yet another advantage of embodiments of the present invention is that the remote monitoring system may be implemented with moderate cost since the remote monitoring system may be incorporated or interfaced with existing sensors and/or an electronic control system of a water treatment system without modification of the design or layout of the water treatment system. Furthermore, the data collected from the water treatment system may be transmitted electronically to the remote computer using, for example, existing communication networks.

According to another broad aspect of the present invention, a method is provided comprising the following steps: (a) transmitting data collected from one or more sensors in the water treatment system to a remote computer disposed at a first distant location from the water treatment system; and (b) generating an output based on the data, wherein the data is transmitted from the water treatment system to the remote computer using a mode of transmission. According to some embodiments, the remote computer may only be connected or linked to the water treatment system via the mode of transmission. According to some embodiments, an analyzer may analyze or manipulate the data to generate the output. The analyzer may comprise a source code or a software program. According to some embodiments, the analyzer may compare the data continuously, in real time, at periodic or selected intervals, on condition, or on demand by a user. According to some embodiments, the output may comprise one or more of the following: data, alarm, analysis result, or analysis report.

According to some of the method embodiments, the water treatment system may comprise a water treatment core facility with the water treatment core facility being a water treatment facility for the distribution of potable drinking water to the public, and the water treatment system may further comprise a distribution system. According to some embodiments, the water treatment system may comprise a water treatment core facility with the water treatment core facility being a wastewater treatment plant (WWTP), and the water treatment system may further comprise a collection system.

According to method embodiments of the present invention, the remote computer may be physically separated from the water treatment system at a distant location, and/or the remote computer may only be connected or linked to the water treatment system via the mode of transmission. According to method embodiments of the present invention, the remote computer itself may comprise may be at least one of the following: a computer, an Internet or web server, a database, or an ftp server. The one or more sensors detect or measure qualities of water in the water treatment system. According to some embodiments, the one or more sensors detect or measure one or more of the following qualities of water in the water treatment system: temperature, chemical composition, total organic carbon (TOC), fluid quantity, flow rate, waste product, contaminant, conductivity, pH, dissolved oxygen, pressure, turbidity, permeate flow, chlorine or fluorine concentration, water or tank level, or equipment status or operation. The one or more sensors may be located at a plurality of locations within the water treatment system. According to some embodiments, at least one of the one or more sensors does not contact the water in the water treatment system. At least one of the one or more sensors not in contact with the water may use radar technology.

According to method embodiments of the present invention, the mode of transmission may vary and may be via one or more of the following: the Internet, TCP/IP, Ethernet, file transfer protocol (ftp), email, such as SMTP, cellular phone network, radios or remote terminal units (RTU) coupled to radio frequency transmitters, satellite transmission, existing telephone or communication networks or wiring, a standard Public Switched Telephone Network (PSTN), a wireless network, a wide area network (WAN), wireless local area network (WLAN), local area network (LAN), or metropolitan area network (MAN), a cable internet connection, short message system (SMS), or a dial-up modem. See description above including additional examples of a mode of transmission. According to some embodiments of the present invention, the data may be transmitted from the water treatment system to the remote computer continuously, in real time, at periodic or selected intervals, on condition, or on demand by a user using the mode of transmission. The data may be transmitted directly from the one or more sensors to the remote computer using a mode of transmission.

Method embodiments of the present invention may further comprise the step of (c) comparing, analyzing, manipulating, etc., the data using an analyzer. According to some embodiments, the manipulating step (c) may comprise comparing the data to expected or historical data or information and/or comparing the data continuously, in real time, at periodic or selected intervals, on condition, or on demand by a user. According to some embodiments, step (c) may further comprise manipulating the data as well as any other information or data, such as historical data, expected performance, etc. to generate an output.

According to some embodiments, the output may comprise one or more of the following: data, an alarm, an analysis result, and/or an analysis report. According to some embodiments, the manipulating step (c) may be performed after the transmitting step (a). According to these embodiments, the analyzer may be located at a second distant location from the water treatment system. According to these embodiments, the first and second distant locations may also be co-located. According to some embodiments, the analyzer may be associated with the remote computer of the remote monitoring system. According to some of these embodiments, the analyzer may be located on the remote computer.

According to embodiments of the present invention, the water treatment system may include a local computer located at or near the water treatment system. According to some embodiments, the data may be transmitted from the local computer located at or near the water treatment system to the remote computer. According to some embodiments, the manipulating step (c) may be performed prior to the transmitting step (a). The local computer may be a logger device. According to these embodiments, the analyzer may be located on the logger device. The logger device may have one or more sensor ports for receiving data from the one or more sensors. The data transmitted from the local computer to the remote computer may include observational data. According to some embodiments, the analyzer may be associated with or on the local computer of the remote monitoring system. Thus, according to some embodiments, the data may be transmitted from the water treatment system by the remote computer accessing the data from the water treatment system, such as the one or more sensors, the electronic control system, and/or the local computer.

According to some method embodiments of the present invention, the water treatment system may include an electronic control system. The electronic control system may be a Supervisory Control and Data Acquisition System (SCADA) or a Progammable Logic Controller (PLC). According to some embodiments, the data may be transmitted from the electronic control system to the remote computer using the mode of transmission.

Method embodiments of the present invention may further comprise the step of (d) communicating the output to a remote viewing device using a mode of communication, wherein step (d) is performed after the generating step (b). According to some embodiments, the output may be accessed from the remote computer or database by a remote viewing device. The remote viewing device may be one or more of the following: personal computer or terminal, web or Internet server, file transfer protocol (ftp) server, cell phone, pager, or handheld device. According to some embodiments, the output may be downloaded or viewed using the remote viewing device. According to some embodiments, the output may be sent or uploaded to the remote viewing device continuously, in real time, at periodic or selected intervals, on condition, or on demand by a user using the mode of communication. The mode of communication may be one or more of the following: Internet, facsimile, file transfer protocol (ftp), voice or text messaging, text to voice messages, electronic mail, pager, human voice calling, SMS messages, instant messaging or groupware protocols, public switched telephone network, cellular network, wireless or satellite communication, or radio communication. See description above including additional examples of a mode of communication. For example, a user viewing the output communicated in step (d) on a remote viewing device may be any one or more of the following: regulator, law enforcement officer, elected official, manager or operator of a water treatment system, vendor customer, member of the public, etc. According to some embodiments, the output may be communicated or submitted to a regulatory and/or law enforcement agency in step (d).

Method embodiments of the present invention may further comprise the step of (e) storing the data on a remote database associated with the remote computer, wherein step (e) may be performed after the generating step (b). According to some embodiments, step (e) may be performed after the manipulating step (c) and/or prior to the communicating step (d).

According to another broad aspect of the present invention, a method is provided for monitoring a water treatment system comprising the following steps: (a) collecting data from one or more sensors located in the water treatment system; and (b) transmitting the data to a remote computer disposed at a first distant location from the water treatment system using a mode of transmission. According to some embodiments, the method may further comprise the step of (c) generating an output based on the data, wherein step (c) is performed after the transmitting step (b). According to some embodiments, the method may further comprise the step of (d) communicating the output to a remote viewing device using a mode of communication, wherein step (d) is performed after the transmitting step (b).

Method embodiments of the present invention may further comprise the step of (e) manipulating the data using an analyzer. According to some embodiments, step (e) is performed prior to step (b). According to these embodiments, the analyzer may be associated with a local computer. According to other embodiments, step (e) may be performed after the transmitting step (b). According to these embodiments, the analyzer may be associated with the remote computer.

Having described many embodiments of the present invention, it will be apparent that modifications, variations, alterations, and changes are possible without departing from the full scope of the invention as defined in the appended claims, and equivalents thereof. It should be appreciated that all examples in the present disclosure, while illustrating many embodiments of the invention, are provided as non-limiting examples and are, therefore, not to be taken as limiting the various aspects so illustrated.

What is claimed is:

1. A remote monitoring system, comprising:
one or more sensors located within a water treatment system being monitored;
a remote computer disposed at a first distant location from the water treatment system; and
an analyzer for manipulating data obtained from the one or more sensors of the water treatment system,
wherein the data is transmitted from the water treatment system to the remote computer using a mode of transmission, and
wherein the remote computer generates an output from the manipulated data.

2. The remote monitoring system of claim 1, wherein the water treatment system comprises a water treatment core facility, wherein the water treatment core facility is a water treatment facility for the distribution of potable drinking water to the public.

3. The remote monitoring system of claim 2, wherein the water treatment system further comprises a distribution system.

4. The remote monitoring system of claim 1, wherein the water treatment system comprises a water treatment core facility, wherein the water treatment core facility is a wastewater treatment plant (WWTP).

5. The remote monitoring system of claim 4, wherein the water treatment system further comprises a collection system.

6. The remote monitoring system of claim 1, wherein the analyzer is located at a second distant location from the water treatment system.

7. The remote monitoring system of claim 6, wherein said first and second distant locations are co-located.

8. The remote monitoring system of claim 1, wherein the analyzer is associated with the remote computer of the remote monitoring system.

9. The remote monitoring system of claim 8, wherein the analyzer is located on the remote computer.

10. The remote monitoring system of claim 1, wherein the remote computer is only connected or linked to the water treatment system via the mode of transmission.

11. The remote monitoring system of claim 1, wherein the remote computer comprises at least one of the following: a computer, an Internet or web server, a database, or an ftp server.

12. The remote monitoring system of claim 1, wherein the one or more sensors detect or measure qualities of water in the water treatment system.

13. The remote monitoring system of claim 12, wherein the one or more sensors are located at a plurality of locations within the water treatment system.

14. The remote monitoring system of claim 12, wherein the one or more sensors detect or measure one or more of the following qualities of water in the water treatment system: temperature, chemical composition, total organic carbon (TOC), fluid quantity, flow rate or fluid velocity, waste product, contaminant, conductivity, pH, dissolved oxygen, pressure, turbidity, permeate flow, chlorine or fluorine concentration, water or fluid level, or equipment status or operation.

15. The remote monitoring system of claim 1, wherein at least one of the one or more sensors does not contact the water in the water treatment system.

16. The remote monitoring system of claim 15, wherein at least one of the one or more sensors not in contact with the water uses look-down ultrasonic technology.

17. The remote monitoring system of claim 15, wherein at least one of the one or more sensors not in contact with the water uses radar technology.

18. The remote monitoring system of claim 1, wherein the mode of transmission is via one or more of the following: the Internet, TCP/IP, MODBUS RTU, MODBUS ASCII, MODBUS TCP, XML, cellular modem, Bluetooth®, ZigBee®, Ethernet, file transfer protocol (ftp), email, such as SMTP, cellular phone network, such as CDMA and TDMA, radios or remote terminal units (RTU) coupled to radio frequency transmitters, satellite transmission, SDI-12, existing telephone or communication networks or wiring, a standard Public Switched Telephone Network (PSTN), dial-up modem using landline or telephone, a wireless network, such as wi-fi, a wide area network (WAN), wireless local area network (WLAN), local area network (LAN), or metropolitan area network (MAN), a cable internet connection, short message system (SMS), a point to point link; global system for mobile communications (GSM, 3GSM), general packet radio services (GPRS), evolution-data optimized (EV-DO), enhanced data rates for GSM evolution (EDGE), digital enhanced cordless telecommunications (DECT), integrated digital enhanced network (iDEN), universal mobile telecommunications systems (UMTS), or advanced mobile phone systems (AMPS).

19. The remote monitoring system of claim 1, wherein the data is transmitted from the water treatment system to the remote computer continuously, in real time, at periodic or selected intervals, on condition, or on demand by a user using the mode of transmission.

20. The remote monitoring system of claim 1, wherein the data is transmitted directly from the one or more sensors to the remote computer using the mode of transmission.

21. The remote monitoring system of claim 1, wherein the analyzer comprises a source code or a software program.

22. The remote monitoring system of claim 1, wherein the analyzer compares the data to expected or historical data or information.

23. The remote monitoring system of claim 1, wherein the analyzer manipulates the data continuously, in real time, at periodic or selected intervals, on condition, or on demand by a user.

24. The remote monitoring system of claim 1, wherein the output comprises one or more of the following: data, alarm, analysis result, or analysis report.

25. The remote monitoring system of claim 1, wherein the water treatment system includes an electronic control system.

26. The remote monitoring system of claim 25, wherein the electronic control system is a Supervisory Control and Data Acquisition System (SCADA) or a Progammable Logic Controller (PLC).

27. The remote monitoring system of claim 25, wherein the data is transmitted from the electronic control system to the remote computer using the mode of transmission.

28. The remote monitoring system of claim 1, further comprising a remote viewing device, wherein the output is sent or uploaded to the remote viewing device via a mode of communication.

29. The remote monitoring system of claim 28, wherein the remote viewing device is one or more of the following: personal computer or terminal, web or Internet server, file transfer protocol (ftp) server, cell phone, pager, or handheld device.

30. The remote monitoring system of claim 28, wherein the output is sent or uploaded to the remote viewing device continuously, in real time, at periodic or selected intervals, on condition, or on demand by a user using the mode of communication.

31. The remote monitoring system of claim 28, wherein the mode of communication is one or more of the following: Internet, TCP/IP, MODBUS RTU, MODBUS ASCII, MODBUS TCP, XML, Ethernet, facsimile, file transfer protocol (ftp), voice or text messaging, text to voice messages, Bluetooth®, ZigBee®, electronic mail, pager, human voice calling, short message system (SMS) messages, instant messaging or groupware protocols, public switched telephone network (PSTN), cellular network, SDI-12, wireless network, satellite communication, a wide area network (WAN), wireless local area network (WLAN), local area network (LAN), metropolitan area network (MAN), dial-up modem, radio communication, global system for mobile communications (GSM, 3GSM), general packet radio services (GPRS), evolution-data optimized (EV-DO), enhanced data rates for GSM evolution (EDGE), digital enhanced cordless telecommunications (DECT), integrated digital enhanced network (iDEN), universal mobile telecommunications sytems (UMTS), or advanced mobile phone systems (AMPS).

32. The remote monitoring system of claim 1, further comprising a remote database associated with the remote computer for storing the data.

33. The remote monitoring system of claim 1, further comprising a local computer located at or near the water treatment system.

34. The remote monitoring system of claim 33, wherein the analyzer is associated with the local computer of the remote monitoring system.

35. The remote monitoring system of claim 34, wherein the analyzer is located on the local computer.

36. The remote monitoring system of claim 33, wherein the data is transmitted from the local computer to the remote computer using the mode of transmission.

37. The remote monitoring system of claim 36, wherein the data transmitted from the local computer to the remote computer includes observational data.

38. The remote monitoring system of claim 33, wherein the local computer is a logger device.

39. The remote monitoring system of claim 38, wherein the analyzer is located on the logger device.

40. The remote monitoring system of claim 38, wherein the logger device has one or more sensor ports for receiving data from the one or more sensors.

41. The remote monitoring system of claim 38, wherein the logger device is water-tight and enclosed.

42. A method for monitoring a water treatment system comprising the following steps:
(a) transmitting data collected from one or more sensors in the water treatment system to a remote computer disposed at a first distant location from the water treatment system; and
(b) generating an output based on the data, wherein the data is transmitted from the water treatment system to the remote computer using a mode of transmission.

43. The method of claim 42, wherein the water treatment system comprises a water treatment core facility, wherein the water treatment core facility is a water treatment facility for the distribution of potable drinking water to the public.

44. The method of claim 43, wherein the fluid treatment system further comprises a distribution system.

45. The method of claim 42, wherein the water treatment system comprises a water treatment core facility, wherein the water treatment core facility is a wastewater treatment plant (WWTP).

46. The method of claim 45, wherein the water treatment system further comprises a collection system.

47. The method of claim 42, wherein the remote computer is only connected or linked to the water treatment system via the mode of transmission.

48. The method of claim 42, wherein the remote computer comprises at least one of the following: a computer, an Internet or web server, a database, or an ftp server.

49. The method of claim 42, wherein the one or more sensors detect or measure qualities of water in the water treatment system.

50. The method of claim 42, wherein the one or more sensors are located at a plurality of locations within the water treatment system.

51. The method of claim 42, wherein the one or more sensors detect or measure one or more of the following qualities of water in the water treatment system: temperature, chemical composition, total organic carbon (TOC), fluid quantity, flow rate or fluid velocity, waste product, contaminant, conductivity, pH, dissolved oxygen, pressure, turbidity, permeate flow, chlorine or fluorine concentration, water or fluid level, or equipment status or operation.

52. The method of claim 42, wherein at least one of the one or more sensors does not contact the water in the water treatment system.

53. The method of claim 56, wherein at least one of the one or more sensors not in contact with the water uses look-down ultrasonic technology.

54. The method of claim 56, wherein at least one of the one or more sensors not in contact with the water uses radar technology.

55. The method of claim 42, wherein the mode of transmission is via one or more of the following: the Internet, TCP/IP, MODBUS RTU, MODBUS ASCII, MODBUS TCP, XML, cellular modem, Bluetooth®, ZigBee®, Ethernet, file transfer protocol (ftp), email, such as SMTP, cellular phone network, such as CDMA and TDMA, radios or remote terminal units (RTU) coupled to radio frequency transmitters, satellite transmission, SDI-12, existing telephone or communication networks or wiring, a standard Public Switched Telephone Network (PSTN), dial-up modem using landline or telephone, a wireless network, such as wi-fe, a wide area network (WAN), wireless local area network (WLAN), local area network (LAN), or metropolitan area network (MAN), or a cable internet connection, short message system (SMS) a point to point link; global system for mobile communications (GSM, 3GSM), general packet radio services (GPRS), evolution-data optimized (EV-DO), enhanced data rates for GSM evolution (EDGE), digital enhanced cordless telecommunications (DECT), integrated digital enhanced network (iDEN), universal mobile telecommunications systems (UMTS), or advanced mobile phone systems (AMPS).

56. The method of claim 42, wherein the data is transmitted from the water treatment system to the remote computer continuously, in real time, at periodic or selected intervals, on condition, or on demand by a user using the mode of transmission.

57. The method of claim 42, wherein the data is transmitted directly from the one or more sensors to the remote computer using the mode of transmission.

58. The method of claim 42, further comprising the step of (c) manipulating the data using an analyzer.

59. The method of claim 58, wherein the analyzer comprises a source code or a software program.

60. The method of claim 58, wherein the manipulating step (c) comprises comparing the data to expected or historical data or information.

61. The method of claim 58, wherein the analyzer compares the data continuously, in real time, at periodic or selected intervals, on condition, or on demand by a user.

62. The method of claim 58, wherein the manipulating step (c) is performed after step (a).

63. The method of claim 62, wherein the analyzer is located at a second distant location from the water treatment system.

64. The method of claim 63, wherein said first and second distant locations are co-located.

65. The method of claim 64, wherein the analyzer is associated with the remote computer of the remote monitoring system.

66. The method of claim 65, wherein the analyzer is located on the remote computer.

67. The method of claim 42, wherein the water treatment system includes an electronic control system.

68. The method of claim 66, wherein the electronic control system is a Supervisory Control and Data Acquisition System (SCADA) or a Progammable Logic Controller (PLC).

69. The method of claim 66, wherein the data is transmitted from the electronic control system to the remote computer using the mode of transmission.

70. The method of claim 42, wherein the data is transmitted from a local computer to the remote computer during step (a), and wherein the local computer is located at or near the water treatment system.

71. The method of claim 58, wherein step (c) is performed prior to step (a).

72. The method of claim 71, wherein the data is transmitted from a local computer to the remote computer during step (a), and wherein the local computer is located at or near the water treatment system.

73. The method of claim 70, wherein the local computer is a logger device.

74. The method of claim 73, wherein the analyzer is located on the logger device.

75. The method of claim 73, wherein the logger device has one or more sensor ports for receiving data from the one or more sensors.

76. The method of claim 73, wherein the logger device is water-tight and enclosed.

77. The method of claim 72, wherein the data transmitted from the local computer to the remote computer includes observational data.

78. The method of claim 72, wherein the analyzer is associated with the local computer of the remote monitoring system.

79. The method of claim 74, wherein the analyzer is located on the local computer.

80. The method of claim 42, wherein the output comprises one or more of the following: data, alarm, analysis result, or analysis report.

81. The method of claim 42, further comprising the step of (d) communicating the output to a remote viewing device using a mode of communication, wherein step (d) is performed after step (b).

82. The method of claim 70, wherein the remote viewing device is one or more of the following: personal computer or terminal, web or Internet server, file transfer protocol (ftp) server, cell phone, pager, or handheld device.

83. The method of claim 70, wherein the output is downloaded or viewed using the remote viewing device.

84. The method of claim 70, wherein the output is sent or uploaded to the remote viewing device continuously, in real time, at periodic or selected intervals, on condition, or on demand by a user using the mode of communication.

85. The method of claim 70, wherein the mode of communication is one or more of the following: Internet, TCP/IP, MODBUS RTU, MODBUS ASCII, MODBUS TCP, XML, Ethernet, facsimile, file transfer protocol (ftp), voice or text messaging, text to voice messages, Bluetooth®, ZigBee®, electronic mail, pager, human voice calling, short message system (SMS) messages, instant messaging or groupware protocols, public switched telephone network (PSTN), cellular network, SDI-12, wireless network, satellite communication, a wide area network (WAN), wireless local area network (WLAN), local area network (LAN), or metropolitan area network (MAN), dial-up modem, radio communication, global system for mobile communications (GSM, 3GSM), general packet radio services (GPRS), evolution-data optimized (EV-DO), enhanced data rates for GSM evolution (EDGE), digital enhanced cordless telecommunications (DECT), integrated digital enhanced network (iDEN), universal mobile telecommunications sytems (UMTS), or advanced mobile phone systems (AMPS).

86. The method of claim 42, further comprising the step of (e) storing the data on a remote database associated with the remote computer, wherein step (e) is performed after step (b).

87. A method for monitoring a water treatment system comprising the following steps:
(a) collecting data from one or more sensors located in the water treatment system; and
(b) transmitting the data to a remote computer disposed at a first distant location from the water treatment system using a mode of transmission.

88. The method of claim 87, further comprising the step of (c) generating an output based on the data.

89. The method of claim 88, wherein step (c) is performed after step (b).

90. The method of claim 87, further comprising the step of (d) communicating the output to a remote viewing device using a mode of communication, wherein step (d) is performed after step (b).

91. The method of claim 87, further comprising the step of (e) manipulating the data using an analyzer.

92. The method of claim 91, wherein step (e) is performed prior to step (b).

93. The method of claim 92, wherein the analyzer is associated with a local computer.

94. The method of claim 91, wherein step (e) is performed after step (b).

95. The method of claim 94, wherein the analyzer is associated with the remote computer.
